(19) United States
(12) Patent Application Publication
(10) Pub. No.: US 2012/0296227 A1
(43) Pub. Date: Nov. 22, 2012

(54) Electro-myocardial cardiogram (EmCG) Parameter's CSS/RSS for calculating heart performance

(76) Inventors: Guangren Chen, Porter Ranch, CA (US); Qian Wan, Shanghai (CN); Xiaohong Yang, Shanghai (CN)

(21) Appl. No.: 13/385,739

(22) Filed: Mar. 5, 2012

Related U.S. Application Data
(60) Provisional application No. 61/464,438, filed on Mar. 4, 2011.

(51) Int. Cl. A61B 5/0402

(52) U.S. Cl. 600/509

(57) ABSTRACT

A method to evaluate Electro-Myocardial Cardiogram (EmCG) parameters is disclosed. The method comprises calculating a Contraction Strength Strain (CSS) and Relaxation Strength Strain (RSS) index. The calculation is represented by a non-linear waveform that represents the excitement function and pump function of the heart.

### CROSS REFERENCE TO RELATED APPLICATION

61/464,438

### FIELD OF THE INVENTION

The present invention generally relates to measurement of heart conducting systems. More specifically, the present invention relates to measurement of cardiac activity based on non-linear waveforms.

### BACKGROUND OF THE INVENTION

Myocardial Ischemia is a disease that is characterized by a reduced blood supply to the heart muscle, usually due to coronary artery disease. The risk for myocardial ischemia increases with age, drinking, smoking, diabetes and other factors. The symptoms include characteristic like chest pain on exertion and decreased tolerance to exercise.

The diagnosis for myocardial ischemia can be done with an electrocardiogram (ECG), echocardiography, or scintigraphy. The ECG uses an ECG waveform, echocardiography uses an ultrasound of the heart, and scintigraphy uses the uptake of a radionuclide by the heart muscle, to detect changes in the heart. The ECG waveform only provides a linear wave of the non-linear functioning of the heart. It does not provide any specific digital data and quantification outcome to represent heart excitation and pumping. Further, the T segment of the ECG waveform represents ventricular pump range. As the T-segment that read is only as a half Sine wave, the ECG does not provide enough ventricular pump power working information. Moreover, echocardiography or invasive methods do not represent a direct detection of the cardiac energy.

Hence, these methods do not provide detailed information on regional myocardial deformation and the quantitative evaluation of the left ventricular systolic and diastolic function. Further, such methods are also affected by the change in the cardiac myocardium strength (i.e. amount of Ca++ ions released during the excitation sequence of the heart).

So, there is a need of a method of evaluating changes in the heart deformation that addresses the aforementioned problems.

### SUMMARY OF THE INVENTION

It is an object of the invention to provide a new Electro-MyocardialCardiogram (EmCG) parameter that uses a non-linear waveform to indicate the non-linear activity of the heart.

It is an object of the present invention to provide detailed information on regional myocardial deformation.

It is a further object of the present invention to provide a quantitative evaluation of the ventricular diastolic and systolic function.

It is another object of the present invention to provide a method to evaluate data that is free from the impact of cardiac myocardium strength.

It is yet another object of the invention to automate the heart diagnostic system through ECG signal processing and application of computer platforms (by applying principle of conservation of energy and a series of non-linear theory).

### BRIEF DESCRIPTION OF THE DRAWINGS

FIG. 1 shows the structure of a human heart;

FIG. 2 shows a Contraction Strength Strain (CSS) and Relaxation Strength Strain (RSS) region of a heart;

FIG. 3 shows a flow chart representing a method to calculate EmCG parameters, according to the present invention; and

FIG. 4 shows a block diagram of a system to calculate EmCG parameters, according to the present invention.

### DETAILED DESCRIPTION OF THE DRAWINGS

In the detailed description of the invention, numerous specific details are described to provide a thorough understanding of the various embodiments of the invention. However, one skilled in the relevant art will recognize that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the invention.

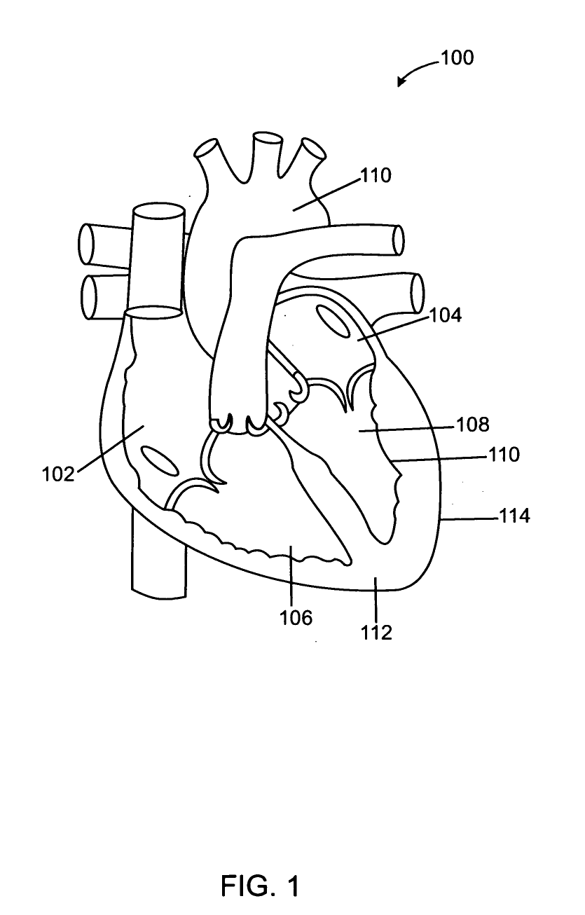

FIG. 1 shows the structure of a human heart 100. The heart 100 comprises four chambers—a right atrium 102, a left atrium 104, a right ventricle 106, and a left ventricle 108. An outer wall of the heart 100 is made of three layers of tissue such as an endocardium 110, a myocardium 112 and an epicardium 114. The myocardium 112 is the muscular wall of the heart 100, or the heart muscle. It contracts to pump blood out of the heart 100 and then relaxes as the heart 100 refills with returning, blood. The endocardium 110 is the innermost layer and is in contact with the blood that the heart 100 pumps. The epicardium 114 is the outermost layer of the heart 100.

The myocardium 112 is the muscular middle layer of the wall of the heart 100. It is composed of spontaneously contracting cardiac muscle fibers that allow the heart 100 to contract. The coordinated contractions of cardiac muscle cells in the heart 100 propel blood out of the two atria (102, 104) and the two ventricles (106, 108) to the blood vessels of the body and the pulmonary circulatory systems. This action makes up the systole or contraction of the heart 100.

Diastole or relaxation is the period of time when the heart 100 fills with blood after the systole (contraction). It is an action opposite systole. Ventricular diastole is the period during which the ventricles (106, 108) are relaxing.

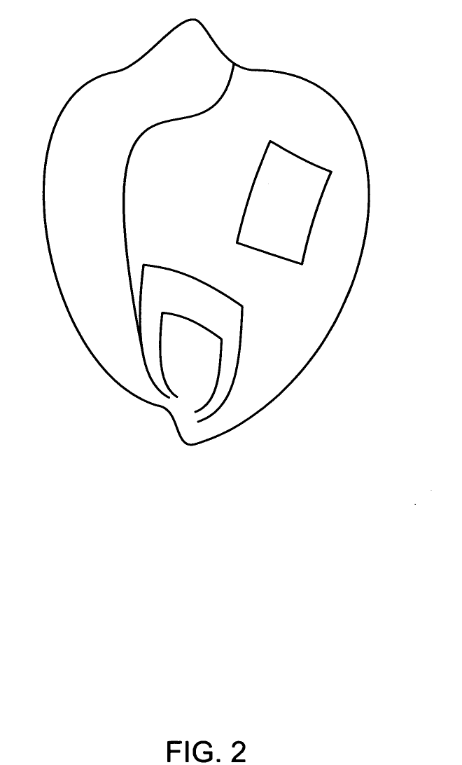

FIG. 2 shows a Contraction Strength Strain (CSS) and Relaxation Strength Strain (RSS) region of the heart 100. CSS and RSS are new Electro-Myocardialcardiogram (EmCG) parameters. EmCG parameters evaluate the two major functions of the heart 100 such as excitement and pump. The EmCG parameters represent the electric data that is generated by biological metabolism after the cardiac muscle does work. This is indicated with segment data in the period from cardiac excitement to pumping, while myocardial tension, length and other factors are not calculated.

CSS and RSS parameters are calculated beat-by-beat by applying myocardial Bio-Energy-Electrodynamics on the basis of work-energy-load. CSS shows the strain observed in myocardium during contraction of the heart whereas RSS shows the strain observed in the myocardium during relaxation of the heart. These parameters are used to represent the myocardial physiological functions of the heart and to forecast the pathological changes.

The CSS and RSS parameters can be calculated by the following empirical formula:

$${lim}_{x \to 0} inf \frac{{xf}^{′} ( x )}{f ( x )}$$

where: lim: limit
   inf: indefinite integral
   ƒ: frequency domain function of the response system
   X: variable (time domain factor)

Frequency domain function ƒ changes with time domain factor x and the change rate varies with point. If ƒ changes at a constant change rate (the change rate at x₀), the value of ƒ under that circumstance will be called the pseudo value of ƒ. The ratio between the pseudo value and the real value of ƒ at point x₀ is calculated. The minimum value of the elements in the set formed by the lower limits of these ratios, i.e. the limit values of all convergent sequences is the desired parameter CSS and RSS.

The parameters CSS and RSS defined by the above stated formula reflect the energy (frequency domain) distribution of the cardiac signal in different dimensional intervals and also reflect the variation and variation rate between different dimensional intervals. Further, it also reflects the velocity change (energy misbalance and load) in time and space interval and represents the energy distribution of cardiac work information in different frequency and time domain intervals.

CSS and RSS parameters help in providing an analysis of heart disease, cardiovascular disease, cardiomyopathy, myocardial metabolic disorder and other diseases.

The value of CSS/RSS for a healthy heart lies between 5 and 10. Any value of the parameter outside this range represents abnormalities of functioning of the heart.

Calculation of the EmCG parameters applies the latest integrated theories, gradually created fractal, chaos, evolution, furcation, nonlinear time and frequency domain, fractal, fractal dimension, reconstructed space and energy sequences and the nonlinear dynamic theories based on the modeling of EmCG series technologies to the advanced medical applications. Multi-dimensional dynamic cardiac information may be extracted from 1D-ECG. This new technology may be added to the conventional ECG which may generate new, effective and more accurate diagnostic methods.

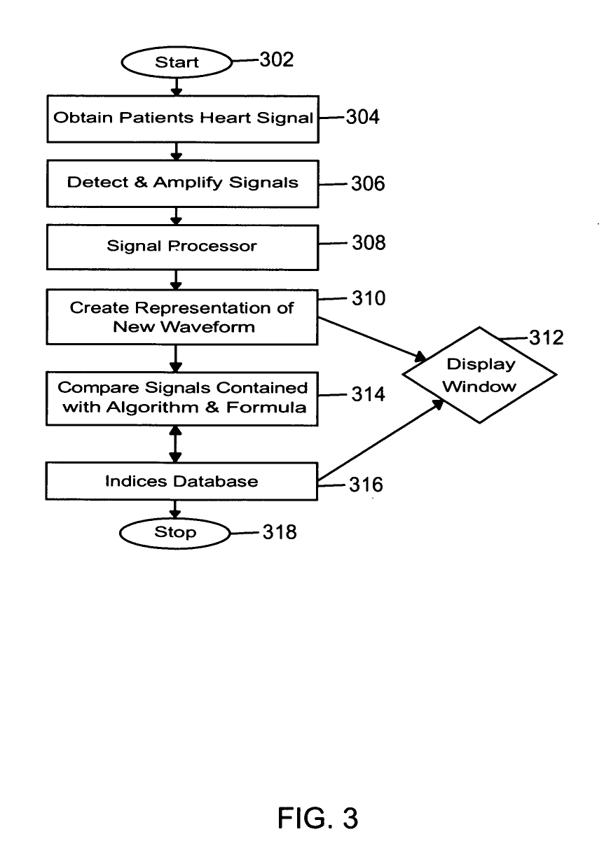

FIG. 3 shows a flow chart representing a method to calculate EmCG parameters, according to the present invention. The method initiates at step 302. At step 304, a patient's heart signal is obtained. This can be done by various methods. Examples of the methods include, but are not limited to ECG, Vital Sign Monitoring, and screen device. These various methods comprise placing electrodes or a wand on various parts of the skin to detect a heart signal. The heart signals thus obtained are detected and amplified at step 306. At step 308, signal processing of the amplified heart signal takes place. This helps in obtaining a multi-domain information (fractal and dimensions) data from the one dimensional ECG signal. The processed signal is then represented by a new waveform at step 310. The new waveform is represented in accordance with various parts of the heart, fractal dimensions and reconstructed phase space. The new waveform thus obtained at step 310, is displayed on a display window at step 312. At step 314, the signal that has been obtained is processed with the algorithm and the parameters formula. The parameters are thus obtained, which on one hand reflect the energy (frequency domain) distribution of cardiac signal in different dimensional intervals, variation and variation rate between different dimensional intervals. On the other hand, the parameters reflect the velocity change (energy misbalance and load) in time and space interval. Further, the parameters reflect the energy distribution of cardiac work information in different time domain intervals and frequency domain interval, and the peak values (indices) of energy regions.

The parameters thus obtained are compared to the indices database at step 316. The indices database contains various indices information of patients who have had at least some form of cardiac disease and patients who have had no cardiac disease. Hence, when the patients EmCG parameters obtained through signal processing and calculation is compared to other indices in the database, a heart abnormality can be identified and diagnosed. This information is then displayed on the display window. The method then terminates at step 318.

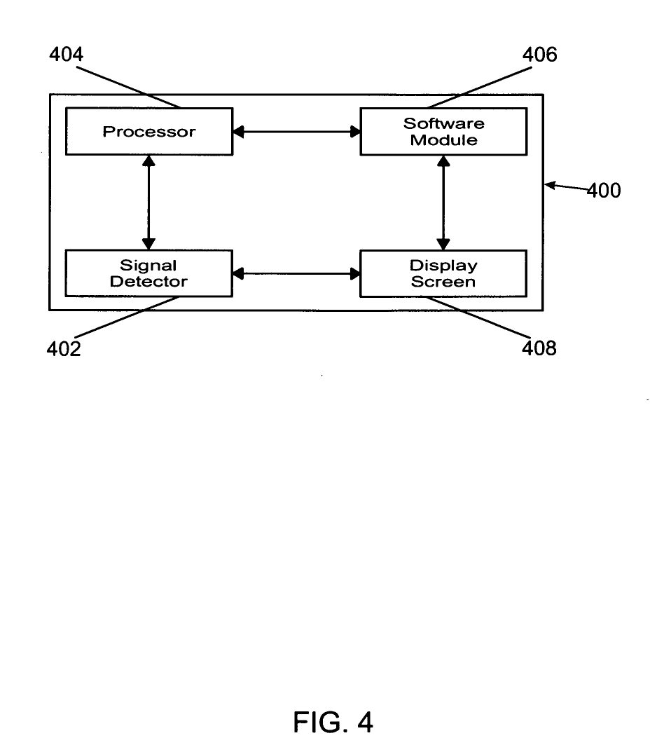

FIG. 4 shows a block diagram of a system to calculate EmCG parameters, according to the present invention. The system 400 comprises a signal detector 402, a processor 404, a software module 406 and a device screen 408. The system 400 should preferably have an interference of 50 Hz and 60 Hz.

Firstly, a signal source of electric physiology is obtained. The signal source is obtained through the signal detector 402. In an embodiment, the system 400 is an Electrocardiogram (ECG) and the signal detector 402 is an electrode or a wand. Various electrodes maybe placed on the skin. The electrical activity of the heart over time is captured and externally recorded by skin electrodes. The recording then maybe produced by the ECG.

The signal detected by the signal detector 402 is then processed by the processor 404. The processing is done by the software module 406, installed in the processor 404. The processor 404 formulates and displays a multi-domain heart signal on the display screen 408. This signal represents information of various parts of the heart, fractal dimensions and reconstructed phase space. The processor 404 then applies an algorithm and formula (described in FIG. 2) to the signal. The indices/parameters CSS and RSS are then calculated and displayed on the screen 408.

In an embodiment of the present invention, the hardware (for example, an ECG) must meet technical as well as indices interface design requirements. The technical requirements include, for example, CMR, sampling rate, anti DC interference, anti EMG interference, 50/60 Hz filter, etc. The algorithm/formula of indices may be included in an indices software package.

Numerous alterations of the processes and the methods herein disclosed will suggest themselves to those skilled in the art. However, it is to be understood that the present disclosure relates to the preferred embodiment of the invention which is for purposes of illustration only and not to be construed as a limitation of the invention.

What is claimed is:

1. A method for evaluating myocardial activity of a heart, the method comprising:
a. obtaining an electrical signal of cardiac activity of the heart;
b. processing said signal to form a multi-dimensional waveform;
c. calculating at least one ElectromyocardialCardiogram (EmCG) parameter using said processed signal, wherein said EmCG parameter is based on a strain strength activity of the heart; and
d. comparing said at least one EmCG parameter with values contained in an indices database to evaluate myocardial activity of the heart.

2. The method of claim 1 further comprises: amplifying said electrical signal using an analog-to-digital processor and a digital-to-analog processor.

3. The method of claim 1, wherein said signal processing further comprises separating said signal into multiple components, wherein the multiple components can be selected from a group consisting of a time, a frequency, an amplitude and a location.

4. The method of claim 1, wherein the values stored in the indices database are measured EmCG parameters with respect to coronary heart disease.

5. The method of claim 1, wherein said calculating further comprises calculating a ratio of a pseudo value and a real value of a frequency domain function (f) of a response system.

6. The method of claim 5 further comprises: calculating a minimum value of elements formed by lower limits of said ratio in time domain to obtain said EmCG parameter, wherein said minimum value is calculated for a range corresponding to a contraction and/or relaxation activity of the heart in a measured ECG signal.

7. The method of claim 5, wherein the frequency domain function (f) changes at a constant change rate.

8. The method of claim 1, wherein said EmCG parameter is calculated based on an equation
$$EmCG parameter = {lim}_{x \to 0} inf \frac{{xf}^{′} ( x )}{f ( x )}$$
wherein, lim represents limit, inf represents indefinite integral, ƒ represents frequency domain function of the response system, ƒ′ represents pseudo value of f, and X represents variable for a time domain factor.

9. The method of claim 1, wherein said EmCG parameter is a Contraction Strain Strength (CSS) of the heart.

10. The method of claim 1, wherein said EmCG parameter is a Relaxation Strain Strength (RSS) of the heart.

11. A method for evaluating myocardial activity of a heart, the method comprising:
a. obtaining an electrical signal of cardiac activity of the heart;
b. processing said signal to form a multi-dimensional waveform;
c. calculating a ratio of a pseudo value and a real value of a frequency domain function (f) of a response system;
d. calculating a minimum value of elements formed by lower limits of said ratio in all time domain to obtain at least one ElectromyocardialCardiogram (EmCG) parameter, wherein said minimum value is calculated for a range corresponding to a contraction and/or relaxation activity of the heart in a measured ECG signal; and
e. comparing said EmCG parameter with values contained in an indices database to evaluate myocardial activity of the heart.

12. A method for evaluating myocardial activity of a heart, the method comprising:
a. obtaining an electrical signal of cardiac activity of the heart;
b. processing said signal to form a multi-dimensional waveform;
c. calculating at least one ElectromyocardialCardiogram (EmCG) parameter using said processed signal, wherein said EmCG parameter is calculated using an equation
$$EmCG parameter = {lim}_{x \to 0} inf \frac{{xf}^{′} ( x )}{f ( x )}$$
wherein, lim represents limit, inf represents indefinite integral, ƒ represents frequency domain function of the response system, ƒ′ represents pseudo value of f, and X represents variable for a time domain factor; and
d. comparing said at least one EmCG parameter with values contained in an indices database to evaluate the myocardial activity of the heart.

13. A system for evaluating myocardial activity of a heart, the system comprising:
a. a signal sensing means for obtaining an electrical signal of the heart;
b. a first processor capable of processing the electrical signal to form a multi-dimensional waveform;
c. a second processor capable of calculating a ratio of a pseudo value and a real value of a frequency domain function (f) of a response system, wherein said second processor is further capable of calculating a minimum value of elements formed by lower limits of said ratio in time domain to obtain said EmCG parameter, wherein said minimum value is calculated for a range corresponding to a contraction and/or relaxation activity of the heart;
d. an indices database containing EmCG parameter values for different heart conditions; and
e. a comparator for comparing said EmCG parameter with values contained in the indices database to evaluate the myocardial activity of the heart.

14. The method of claim 13, wherein the frequency domain function (f) changes at a constant change rate.

15. The system of claim 13, wherein said first processor and said second processor are integrated.

16. The system of claim 13 further comprises: an analog-to-digital processor for amplifying the electrical signal.

17. The system of claim 13 further comprises: a signal separator for separating the processed signal into multiple components, wherein the multiple components can be selected from a group consisting of: a frequency, a time, an amplitude and a location.

18. The system of claim 13, wherein said EmCG parameter is calculated based on an equation, wherein the equation is
$$EmCG parameter = {lim}_{x \to 0} inf \frac{{xf}^{′} ( x )}{f ( x )}$$
wherein, lim represents limit, inf represents indefinite integral, ƒ represents frequency domain function of the response system, ƒ′ represents pseudo value of f, and X represents variable for a time domain factor.

19. The system of claim 13, wherein said EmCG parameter is a Contraction Strain Strength (CSS) of the heart.

20. The system of claim 13, wherein said EmCG parameter is a Relaxation Strain Strength (RSS) of the heart.
